(19) United States
(12) Patent Application Publication
(10) Pub. No.: US 2023/0352116 A1
(43) Pub. Date: Nov. 2, 2023

(54) GROUP OF SINGLE NUCLEOTIDE POLYMORPHISM LOCI AND METHOD FOR IDENTIFYING BIOGEOGRAPHIC ORIGINS OF EAST ASIAN POPULATIONS

(71) Applicant: GUIZHOU MEDICAL UNIVERSITY, Guiyang (CN)

(72) Inventors: Xiaoye JIN, Guiyang (CN); Jiang HUANG, Guiyang (CN); Guiyin ZHOU, Guiyang (CN); Zheng REN, Guiyang (CN); Hongling ZHANG, Guiyang (CN); Qiyan WANG, Guiyang (CN); Yubo LIU, Guiyang (CN); Jingyan JI, Guiyang (CN); Bing XIA, Guiyang (CN)

(21) Appl. No.: 18/306,502

(22) Filed: Apr. 25, 2023

(30) Foreign Application Priority Data
Apr. 28, 2022 (CN) 202210463446.2

(51) Int. Cl. G16B 20/20

(52) U.S. Cl. CPC G16B 20/20

(57) ABSTRACT

Disclosed are a group of single nucleotide polymorphism (SNP) loci and a method for identifying biogeographic origins of East Asian populations, and belong to the technical field of gene identification. The application takes single nucleotide polymorphism molecular genetic markers as objects, systematically selects loci with high genetic differentiation in the East Asian populations of Beijing Han population, Southern Han population, Dai population, Japanese and Kinh Population from Vietnam, and constructs an efficient, simple and fast artificial intelligence model through the XGBoost machine learning algorithm for analyzing biogeographic origins of five East Asian populations.

### CROSS-REFERENCE TO RELATED APPLICATIONS

This application claims a priority to Chinese Patent Application No. 202210463446.2, filed on Apr. 28, 2022, the contents of which are hereby incorporated by reference.

### TECHNICAL FIELD

The application relates to a technical field of gene identification, and in particular to a group of single nucleotide polymorphism (SNP) loci and a method for identifying biogeographic origins of East Asian populations.

### BACKGROUND

Ancestry informative markers refer to genetic markers representing higher allele frequency differences in populations. They may analyze a biogeographic origin of unknown individuals and may also be used to identify potential substructures in a population. The former role may provide directional clues for judicial investigations in forensic medicine research; the latter role may control factors of population stratification in whole-genome association study, so as to avoid false positive or false negative results. At present, forensic scientists usually pay attention to identifications of major intercontinental populations. Up to now, several populations of ancestry informative markers for forensic ancestry analysis of different intercontinental populations have been reported. However, there is relatively little research on the forensic ancestry analysis of populations in a same continent or populations in the major intercontinental populations.

For the analysis of biogeographic origins of unknown individuals, forensic scientists usually use a principal component analysis method or a population genetic structure analysis method. The principal component analysis method performs a dimension-reduction method on all samples according to information of all loci, transforms variable information into several important principal components, each sample has a specific position in the different principal components, and then infers the possible biogeographic origin of the individual according to the distribution of samples in the different principal components. The population genetic structure analysis method estimates a proportion of individual ancestry components based on Bayesian method, and then determines the origin of individual ancestry according to the distribution of ancestry components by comparing with the reference population. However, these two methods may not be able to obtain more accurate prediction results for individuals with mixed history.

Single nucleotide polymorphism (SNP) is a sequence polymorphism formed by the variation of a single nucleotide in the genome. It has advantages of a wide distribution and a low mutation rate in the genome, and has high application value in the forensic research. In addition, previous studies have found that some single nucleotide polymorphisms show high differences in allele frequency distribution among different populations, and may be used as ancestry information markers to analyze the biogeographic origins of different populations.

### SUMMARY

The objective of the application is to provide a group of single nucleotide polymorphism (SNP) loci and a method for identifying biogeographic origins of East Asian populations, so as to solve the problems existing in the prior art, and these loci may be u sed to identify Beijing Han population, Southern Han population, Dai population, Japanese and Kinh Population from Vietnam.

In order to achieve the above objectives, the application provides following schemes:
the application provides an application of a detection reagent of a group of whole-genome single nucleotide polymorphism loci used for identifying biogeographic origins of East Asian populations in preparing a kit for identifying the biogeographic origins of the East Asian populations, where the single nucleotide polymorphism loci include the loci shown in a following table:

| Chromosome | rs number | Position | Allele 1 | Allele 2 |
|---|---|---|---|---|
| 1 | rs6594028 | 564598 | G | A |
| 1 | rs1801133 | 11856378 | A | G |
| 1 | rs12038287 | 11895396 | C | T |
| 1 | rs561510556 | 12387655 | A | G |
| 1 | rs144246431 | 19674993 | G | T |
| 1 | rs202129706 | 22315762 | A | C |
| 1 | rs140295961 | 33068395 | A | G |
| 1 | rs12731453 | 36676712 | T | G |
| 1 | rs117115434 | 56279497 | A | G |
| 1 | rs576196822 | 62612083 | T | C |
| 1 | rs532154984 | 65314266 | T | C |
| 1 | rs56270653 | 83804841 | C | G |
| 1 | rs552858520 | 84679675 | A | T |
| 1 | rs77172129 | 98602316 | G | A |
| 1 | rs147226864 | 121471638 | T | C |
| 1 | rs6692177 | 143543213 | A | G |
| 1 | rs200220063 | 152882512 | G | A |
| 1 | rs183624843 | 156665281 | T | C |
| 1 | rs16840204 | 158435927 | A | C |
| 1 | rs75985579 | 158988992 | A | G |
| 1 | rs75735370 | 187472432 | G | A |
| 1 | rs7530988 | 205558200 | G | A |
| 1 | rs151191827 | 229641396 | A | G |
| 1 | rs12726054 | 233623860 | A | G |
| 2 | rs77944863 | 3225405 | A | G |
| 2 | rs551794229 | 5162546 | A | G |
| 2 | rs187901830 | 32048491 | G | T |
| 2 | rs530416094 | 39536678 | A | G |
| 2 | rs75837024 | 48763333 | G | A |
| 2 | rs80297078 | 68051286 | C | T |
| 2 | rs557609484 | 92310281 | T | C |
| 2 | rs56339353 | 92320508 | C | A |
| 2 | rs114979404 | 97613974 | G | C |
| 2 | rs189257511 | 97718250 | T | A |
| 2 | rs143319605 | 103166662 | C | T |
| 2 | rs55935451 | 147238877 | A | T |
| 2 | rs55868911 | 177272945 | A | G |
| 2 | rs117736789 | 177439091 | C | G |
| 2 | rs537631083 | 210638066 | A | G |
| 2 | rs146508123 | 226363646 | T | C |
| 3 | rs59692692 | 13571964 | A | T |
| 3 | rs142773888 | 14414901 | T | C |
| 3 | rs144955067 | 31628063 | T | G |
| 3 | rs80350736 | 61914553 | T | C |
| 3 | rs79961039 | 68328083 | C | T |
| 3 | rs73107449 | 69415703 | C | T |
| 3 | rs77486591 | 69513520 | T | A |
| 3 | rs570435573 | 86028382 | T | G |
| 3 | rs544325853 | 97279356 | G | T |
| 3 | rs6778948 | 150134304 | G | A |
| 3 | rs11706245 | 150193109 | G | A |
| 3 | rs9844691 | 150250537 | C | A |
| 3 | rs116783706 | 152553769 | T | C |
| 3 | rs112658986 | 175079928 | C | A |
| 3 | rs575001940 | 183674928 | A | G |
| 3 | rs79806084 | 187520132 | C | T |
| 4 | rs142462241 | 9123223 | C | T |
| 4 | rs370496197 | 9240814 | T | C |
| 4 | rs546642722 | 17813761 | A | G |
| 4 | rs76753571 | 38787305 | G | A |
| 4 | rs5743592 | 38803063 | G | A |
| 4 | rs55750794 | 38851296 | T | C |
| 4 | rs55718051 | 38906717 | G | A |
| 4 | rs7680508 | 100445282 | G | A |
| 4 | rs9884555 | 120869851 | G | T |
| 4 | rs1425419 | 124565964 | T | C |
| 4 | rs280603 | 129915063 | C | A |
| 4 | rs17682978 | 137834738 | C | G |
| 5 | rs201981916 | 1025907 | T | C |
| 5 | rs12658612 | 31238976 | T | G |
| 5 | rs370349765 | 37295709 | T | C |
| 5 | rs78369336 | 41181491 | T | G |
| 5 | rs145999897 | 49432282 | A | G |
| 5 | rs28834498 | 49436826 | G | A |
| 5 | rs75712375 | 65307199 | A | T |
| 5 | rs3850651 | 88181109 | G | T |
| 5 | rs10066711 | 88190604 | T | A |
| 5 | rs117108524 | 88780333 | T | G |
| 5 | rs62381226 | 138366518 | T | C |
| 5 | rs4912927 | 142951094 | A | G |
| 5 | rs74562701 | 172998005 | A | G |
| 6 | rs75585369 | 5138833 | G | A |
| 6 | rs74567382 | 6183479 | A | G |
| 6 | rs56091651 | 14009167 | A | G |
| 6 | rs184103375 | 38488488 | T | C |
| 6 | rs62412779 | 58774684 | G | A |
| 6 | rs7766881 | 82802644 | C | A |
| 6 | rs2815293 | 96769927 | T | C |
| 6 | rs9480779 | 107836678 | C | T |
| 6 | rs565359437 | 108108169 | A | G |
| 6 | rs9402549 | 134239300 | C | T |
| 6 | rs4464817 | 138340676 | A | G |
| 6 | rs535319466 | 152588967 | G | C |
| 6 | rs9457053 | 165622609 | A | G |
| 6 | rs112864719 | 169342074 | A | C |
| 6 | rs75191948 | 170619277 | A | G |
| 7 | rs535914822 | 42834578 | G | C |
| 7 | rs141756608 | 50275516 | T | C |
| 7 | rs200588960 | 61794552 | T | A |
| 7 | rs374938140 | 61794862 | C | T |
| 7 | rs6958030 | 66457975 | C | T |
| 7 | rs76950224 | 130932529 | G | C |
| 7 | rs60560877 | 134697870 | A | G |
| 7 | rs10269898 | 141790229 | G | A |
| 7 | rs3778922 | 151802332 | T | G |
| 8 | rs144799228 | 4172014 | C | T |
| 8 | rs187561464 | 9673968 | A | G |
| 8 | rs117900444 | 32351714 | G | A |
| 8 | rs199569147 | 43825355 | G | T |
| 8 | rs62497902 | 46846688 | A | G |
| 8 | rs372912309 | 46846701 | A | C |
| 8 | rs77994895 | 80546112 | A | T |
| 8 | rs78475651 | 106445484 | G | C |
| 8 | rs80311821 | 119297519 | C | T |
| 8 | rs117673129 | 121843399 | A | G |
| 8 | rs4523256 | 123206335 | C | T |
| 8 | rs77058162 | 123624226 | C | T |
| 8 | rs117059004 | 123765817 | A | G |
| 8 | rs4736545 | 133114957 | A | C |
| 8 | rs2976388 | 143760256 | A | G |
| 9 | rs10816006 | 8937989 | G | T |
| 9 | rs1359095 | 10276100 | C | T |
| 9 | rs7039736 | 29819149 | A | G |
| 9 | rs117745218 | 34851653 | T | C |
| 9 | rs118138111 | 35388117 | C | T |
| 9 | rs117359308 | 44239346 | A | G |
| 9 | rs62547870 | 68396587 | C | T |
| 9 | rs117532342 | 123007609 | C | A |
| 9 | rs10760415 | 128892050 | A | G |
| 9 | rs3780712 | 132943082 | A | G |
| 10 | rs116843849 | 14693330 | T | C |
| 10 | rs58098705 | 25499954 | A | G |
| 10 | rs74213410 | 42399151 | A | T |
| 10 | rs192073133 | 43427620 | T | C |
| 10 | rs2339711 | 53048696 | G | A |
| 10 | rs1649994 | 80070687 | C | G |
| 10 | rs576091513 | 101292805 | G | T |
| 10 | rs75509020 | 134369277 | C | G |
| 11 | rs2071118 | 2972439 | T | C |
| 11 | rs4757893 | 20133413 | G | A |
| 11 | rs145321302 | 34240293 | C | G |
| 11 | rs12785447 | 38438330 | C | G |
| 11 | rs149709595 | 44840723 | C | T |
| 11 | rs1484393 | 45024657 | G | A |
| 11 | rs117641284 | 47248190 | G | A |
| 11 | rs11039176 | 47339169 | G | A |
| 11 | rs10838794 | 48054573 | T | C |
| 11 | rs11039516 | 48124157 | A | T |
| 11 | rs7941996 | 50496359 | T | C |
| 11 | rs147042619 | 60956757 | A | G |
| 11 | rs117682486 | 61015168 | C | T |
| 11 | rs11230736 | 61304473 | C | T |
| 11 | rs143362806 | 61375236 | G | T |
| 11 | rs520987 | 61521446 | C | A |
| 11 | rs7394579 | 61581450 | A | G |
| 11 | rs7394739 | 69692121 | T | C |
| 11 | rs74355568 | 114324060 | T | A |
| 11 | rs10891749 | 114647037 | C | T |
| 11 | rs80253223 | 118722457 | A | C |
| 11 | rs117608910 | 118741152 | C | T |
| 11 | rs189120206 | 119197644 | A | G |
| 11 | rs79626515 | 119980685 | A | G |
| 11 | rs11223547 | 133528942 | A | T |
| 12 | rs3217805 | 4388084 | G | C |
| 12 | rs429561 | 52835321 | C | G |
| 12 | rs77994613 | 54618848 | C | T |
| 12 | rs11170914 | 54861704 | C | T |
| 12 | rs10506426 | 61775492 | C | A |
| 12 | rs536701895 | 75343015 | A | G |
| 12 | rs79705698 | 88508258 | C | T |
| 12 | rs78062178 | 89304157 | G | A |
| 12 | rs11105124 | 89375909 | A | T |
| 12 | rs10860945 | 103539215 | C | T |
| 12 | rs11066427 | 113263909 | G | C |
| 12 | rs11608584 | 128051560 | T | C |
| 13 | rs7328200 | 28615133 | A | G |
| 13 | rs74984577 | 102518262 | T | A |
| 13 | rs540356754 | 113541917 | G | C |
| 14 | rs182863287 | 22445293 | C | T |
| 14 | rs2042518 | 76166481 | T | C |
| 14 | rs78964863 | 89771738 | G | C |
| 14 | rs144885709 | 95893762 | A | T |
| 14 | rs538254210 | 96938945 | T | A |
| 14 | rs77313258 | 101788844 | T | C |
| 14 | rs189231680 | 105862413 | A | T |
| 14 | rs77597431 | 106029023 | T | A |
| 14 | rs8003259 | 106063104 | T | G |
| 14 | rs4983473 | 106081193 | T | C |
| 14 | rs61985604 | 106085447 | C | T |
| 14 | rs75889359 | 106117651 | G | T |
| 14 | rs28720689 | 106127912 | G | A |
| 14 | rs10150934 | 106129418 | T | C |
| 14 | rs2516751 | 106143806 | G | A |
| 14 | rs7494172 | 106175202 | T | C |
| 14 | rs372579409 | 106185689 | C | G |
| 14 | rs186911060 | 106187159 | G | C |
| 14 | rs17841089 | 106207725 | C | T |
| 14 | rs12880412 | 106207805 | C | G |
| 14 | rs61983938 | 106210814 | T | C |
| 14 | rs140451109 | 106225946 | G | C |
| 14 | rs61985395 | 106231158 | G | A |
| 14 | rs2879250 | 106235419 | C | T |
| 14 | rs15979 | 106235489 | T | C |
| 14 | rs1051112 | 106235611 | A | T |
| 14 | rs149653267 | 106235742 | C | G |
| 14 | rs12101008 | 106340358 | T | A |
| 15 | rs12050504 | 25118733 | C | T |
| 15 | rs8038186 | 56095508 | A | G |
| 15 | rs117054397 | 60472480 | A | G |
| 15 | rs370188878 | 60756638 | G | A |
| 15 | rs2439424 | 66979943 | A | G |
| 15 | rs536189723 | 74326699 | C | T |
| 15 | rs558029138 | 101098151 | C | A |
| 16 | rs570636147 | 16452036 | C | T |
| 16 | rs4275872 | 46410819 | G | A |
| 16 | rs543086096 | 46417894 | A | G |
| 16 | rs9285998 | 46426086 | G | A |
| 16 | rs17822931 | 48258198 | C | T |
| 16 | rs7185374 | 48450368 | C | A |
| 16 | rs148106276 | 87864696 | T | C |
| 16 | rs55799444 | 90107716 | T | C |
| 17 | rs76007934 | 2371207 | C | G |
| 17 | rs142708997 | 21965750 | T | C |
| 17 | rs141797564 | 22253602 | T | G |
| 17 | rs202121576 | 22261435 | C | T |
| 17 | rs79399637 | 22261755 | G | T |
| 17 | rs139316749 | 22262103 | T | A |
| 17 | rs78261308 | 36778892 | C | A |
| 17 | rs75060014 | 41038677 | A | G |
| 17 | rs147994591 | 45627005 | A | G |
| 17 | rs140713446 | 46124685 | C | G |
| 17 | rs140900296 | 47089580 | G | T |
| 17 | rs6501525 | 70218627 | A | G |
| 17 | rs77039319 | 70278839 | A | G |
| 17 | rs189618173 | 73722924 | T | C |
| 18 | rs545537217 | 18518431 | T | G |
| 18 | rs6567282 | 60094992 | C | T |
| 19 | rs8100854 | 10720886 | A | T |
| 19 | rs10408721 | 10758319 | T | C |
| 19 | rs138357154 | 17601811 | T | C |
| 19 | rs12986064 | 54755133 | C | T |
| 19 | rs624315 | 54755636 | T | C |
| 19 | rs377681 | 54766423 | A | G |
| 19 | rs1808548 | 54781509 | T | C |
| 19 | rs798899 | 54800767 | T | C |
| 20 | rs6117562 | 753310 | G | A |
| 20 | rs6140211 | 773680 | G | A |
| 20 | rs565751489 | 5547557 | T | A |
| 20 | rs118072189 | 26292074 | T | G |
| 21 | rs59142554 | 35544523 | A | G |
| 21 | rs549950103 | 38533018 | A | T |
| 21 | rs114285135 | 41457206 | C | A |
| 22 | rs540495340 | 20663250 | A | C |
| 22 | rs148969952 | 30958591 | G | C |
| 22 | rs57437434 | 37373430 | A | C |
| 22 | rs138225077 | 42121201 | T | C |
| 22 | rs117410509 | 48654537 | T | C |
| 22 | rs551265777 | 49277658 | G | C |

Optionally, the biogeographic origins of the East Asian populations include Beijing Han population, Southern Han population, Dai population, Japanese and Kinh Population from Vietnam.

The application also provides a method for analyzing the biogeographic origins of the East Asian populations, including steps of screening the group of whole-genome single nucleotide polymorphism loci for identifying the biogeographic origins of the East Asian populations.

Optionally, the steps are as follows:
(1) preliminarily screening relatively highly differentiated single nucleotide polymorphism loci in the East Asian populations by using a PLINK software system based on whole-genome data of the East Asian populations in an international 1000 genomes project; and
(2) using an XGBoost machine learning algorithm, re-screening the single nucleotide polymorphism loci preliminarily screened in the step (1) based on an optimal subset method, and finally determining the single nucleotide polymorphism loci used to analyze the biogeographic origins of the East Asian populations.

Optionally, in the step (1), principles of preliminarily screening relatively highly differentiated single nucleotide polymorphism loci in the East Asian populations include:
(1) fixed coefficients of the Japanese and a non-Japanese population greater than 0.2;
(2) fixed coefficients of the Beijing Han population and the Southern Han population greater than 0.06;
(3) fixed coefficients of the Dai population and the Kinh Population from Vietnam greater than 0.06;
(4) fixed coefficients of a Han population, the Dai population, and the Kinh Population greater than 0.06;
(5) a minimum allele frequency of selected single nucleotide polymorphism loci in each population greater than 0.01;
(6) the selected single nucleotide polymorphism loci consistent with Hardy-Weinberg equilibrium (HWE) in each population, and the P value is greater than 0.0001; and
(7) paired r² of the selected single nucleotide polymorphism loci is less than 0.6.

Optionally, using a principal component analysis method to evaluate an analytic efficiency of the single nucleotide polymorphism loci preliminarily screened in the step (1) on the East Asian populations is further included between the step (1) and the step (2).

Optionally, the following is further included: constructing a prediction model by using the single nucleotide polymorphism loci obtained in the step (2) by re-screening, and evaluating an identification efficiency on the biogeographic origins of the East Asian populations.

The application also provides an application of a group of whole-genome single nucleotide polymorphism loci used for identifying the biogeographic origins of the East Asian populations in forensic medicine and population genetics researches.

The application discloses following technical effects:

The application provides a group of single nucleotide polymorphism loci with high genetic differentiation in the East Asian populations. Compared with the previous different intercontinental populations, the loci in the application may be well used to analyze the biogeographic origins of the East Asian populations, which may provide more valuable information for forensic medicine and population genetics researches.

The application provides a method for analyzing the biogeographic origins of the East Asian populations based on the single nucleotide polymorphism loci. Compared with the conventional methods of principal component analysis and population genetic structure analysis, the method disclosed in the application is simple, fast, accurate and easy to interpret.

### BRIEF DESCRIPTION OF THE DRAWINGS

In order to explain the embodiments of the application or the technical scheme in the prior art more clearly, the drawings needed in the embodiments are briefly introduced below. Obviously, the drawings described below are only some embodiments of the application, and other drawings may be obtained according to these drawings without creative work for ordinary people in the field.

FIG. 1 is a flow chart of single nucleotide polymorphism (SNP) loci screening.

FIG. 2A shows a principal component analysis of five East Asian populations based on the whole-genome single nucleotide polymorphism loci,

FIG. 2B shows a principal component analysis of the five East Asian populations based on the selected 677 single nucleotide polymorphism loci; where CDX: Dai population; CHB: Beijing Han population; CHS: Southern Han population; JPT: Japanese; KHV: Kinh Population from Vietnam.

FIG. 3A shows a confusion matrix diagram of predicted results and actual results for five East Asian populations by the XGBoost based on 677 single nucleotide polymorphism loci.

FIG. 3B shows a confusion matrix diagram of predicted and actual results for five East Asian populations by the XGBoost based on 258 single nucleotide polymorphism loci; where CDX: Dai population; CHB: Beijing Han population; CHS: Southern Han population; JPT: Japanese; KHV: Kinh Population from Vietnam.

### DETAILED DESCRIPTION

A number of exemplary embodiments of the application are described in detail now, and this detailed description should not be considered as a limitation of the application, but should be understood as a more detailed description of certain aspects, characteristics and embodiments of the application.

It should be understood that the terminology described in the application is only for describing specific embodiments and is not used to limit the application. In addition, for the numerical range in the application, it should be understood that each intermediate value between the upper limit and the lower limit of the range is also specifically disclosed. The intermediate value within any stated value or stated range and every smaller range between any other stated value or intermediate value within the stated range are also included in the application. The upper and lower limits of these smaller ranges may be independently included or excluded from the range.

Unless otherwise specified, all technical and scientific terms used herein have the same meaning as commonly understood by one of ordinary skill in the art to which this application relates. Although the application only describes the preferred methods and materials, any methods and materials similar or equivalent to those described herein may also be used in the practice or testing of the application. All documents mentioned in this specification are incorporated by reference to disclose and describe methods and/or materials related to the documents. In case of conflict with any incorporated document, the contents of this specification shall prevail.

It is obvious to those skilled in the art that many improvements and changes may be made to the specific embodiments of the application without departing from the scope or spirit of the application. Other embodiments are apparent to the skilled person from the description of the application. The specification and example of this application are only exemplary.

The terms “including”, “comprising”, “having” and “containing” used in the application are all open terms, which means including but not limited to.

Embodiment 1 A Method for Analyzing Biogeographic Origins of East Asian Populations

The software used in the application mainly includes PLINK, YModel and R software, and are used for screening single nucleotide polymorphism (SNP) loci for identifying biogeographic origins of five East Asian populations: Beijing Han population, Southern Han population, Dai population, Japanese and Kinh Population from Vietnam.

Firstly, preliminarily screening relatively highly differentiated single nucleotide polymorphism loci of five East Asian populations: downloading the whole-genome data of the East Asian populations from the international 1000 genomes; using PLINK software, inputting following codes: ‘plink--bfile all--hwe 0.0001-- maf 0.01--make-bed--out new’, and based on all East Asian individuals, excluding those single nucleotide polymorphism loci whose P value of HWE is less than 0.0001 and the minimum allele frequency is less than 0.01; then using ‘plink--bfile new--indep--pairwise 50 5 0.6’ to keep those single nucleotide polymorphism loci with paired r² values less than 0.6; using ‘plink--bfile new3--within pop.txt--fst’ to calculate a fixed coefficient of each locus in the East Asian populations, and select single nucleotide polymorphism loci with fixed coefficients>0.06; eliminating those loci located in major histocompatibility complex (MHC) region; re-screening the following loci to select those loci with high genetic differentiation among paired populations, and the specific principles are as follows:
1) fixed coefficients of the Japanese and a non-Japanese population are greater than 0.2;
2) fixed coefficients of the Beijing Han population and the Southern Han population are greater than 0.06;
3) fixed coefficients of the Dai population and the Kinh Population from Vietnam are greater than 0.06; and
4) fixed coefficients of a Han population, the Dai population, and the Kinh Population from Vietnam are greater than 0.06.

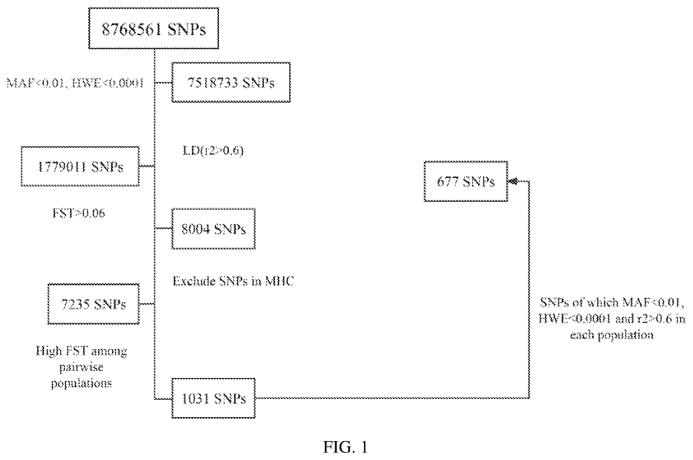

Finally, screening again the above loci by using ‘plink--bfile all--hwe 0.0001--maf 0.01--within pop.txt--make-bed--out new’ and ‘plink--bfile new--within pop.txt--indep- pairwise 50 5 0.6’, and eliminating the single nucleotide polymorphism loci with the P value of HWE less than 0.0001, the minimum allele frequency less than 0.01 and the paired r² value greater than 0.6 in each population. Finally, the application retains 677 single nucleotide polymorphism loci. A flow chart of the above-mentioned single nucleotide polymorphism loci screening is shown in FIG. 1.

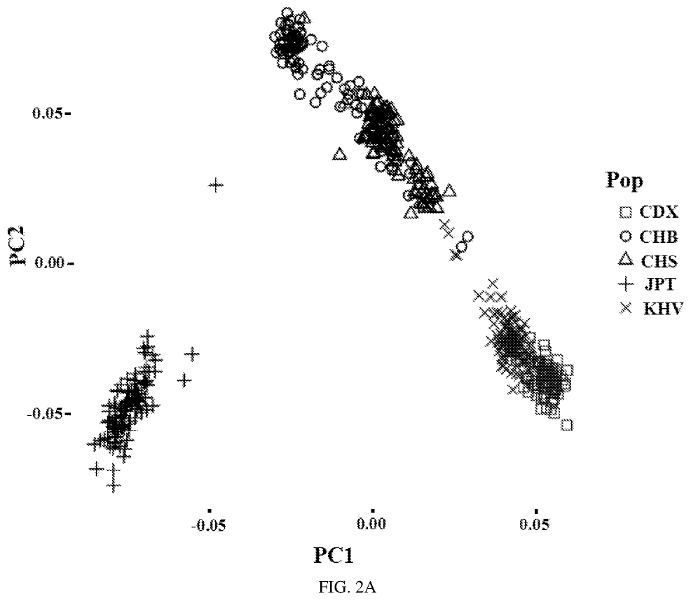

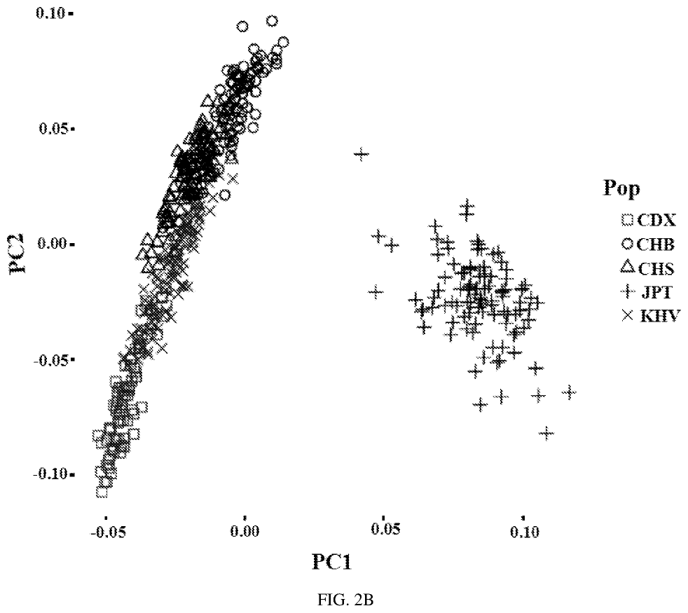

Next, the principal component analysis method commonly used at present is adopted to evaluate the analytic efficiency of 677 single nucleotide polymorphism loci for the East Asian populations. The specific operation are as follows:
carrying out the principal component analysis method on five East Asian populations by using PLINK software, and the code is ‘plink--bfile new1--pca5--out new1’; according to the obtained results, drawing scatter plots of all individuals on the first two principal components by R software. In addition, the principal component analysis is also performed for all loci. Results of principal component analysis of different loci are shown in FIG. 2. The results show that the 677 single nucleotide polymorphism loci selected by the application may reach a population identification level similar to that of the whole-genome loci.

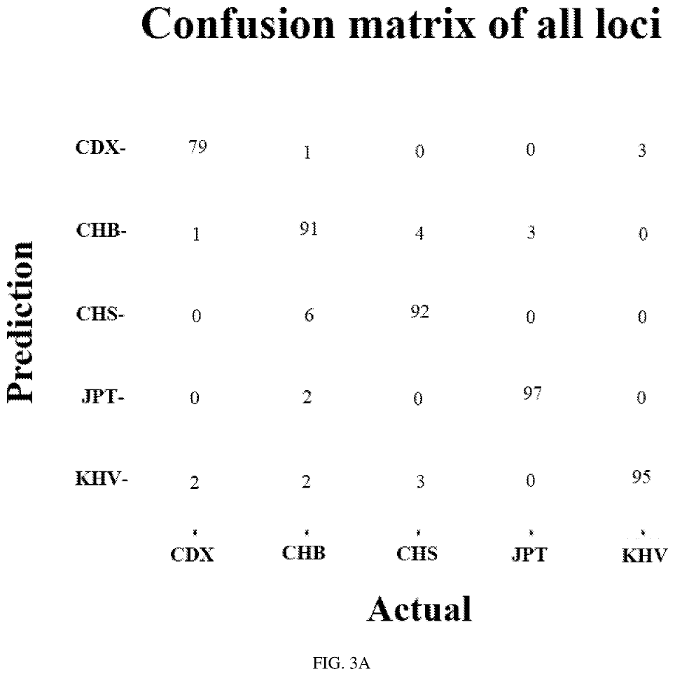

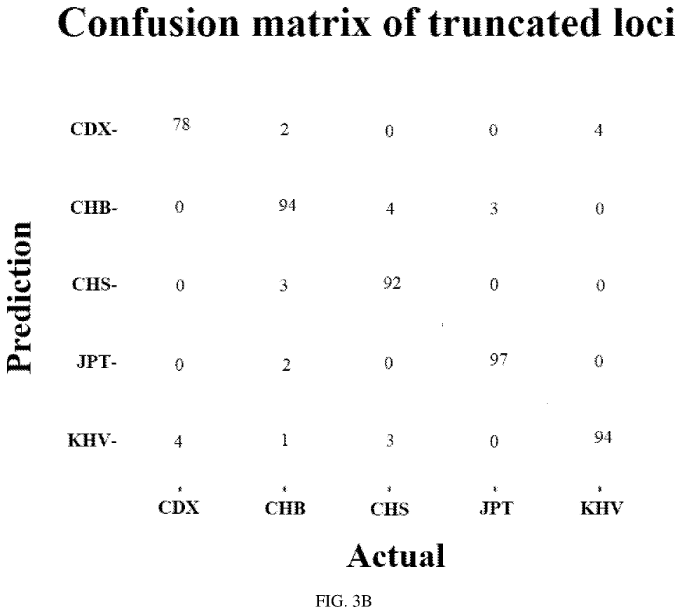

Re-screening of 677 single nucleotide polymorphism loci: the application adopts a machine learning algorithm XGBoost, and re-screens 677 single nucleotide polymorphism loci by using an optimal subset method, and finally determines 258 single nucleotide polymorphism loci. Using 677 and 258 single nucleotide polymorphism loci to construct prediction models respectively, evaluating an identification efficiency on the biogeographic origins of the East Asian populations, and confusion matrix of the predicted results and the actual sample results are shown in FIG. 3A and FIG. 3B. Accuracies and Kappa coefficients of the predicted results and the actual results of the models constructed at different loci are shown in Table 1. The results show that the finally determined 258 single nucleotide polymorphism loci have similar performance in analyzing the biogeographic origins of these five East Asian populations compared with the selected 677 single nucleotide polymorphism loci.

TABLE 1
Comparison of identification performances of 677 and 258 single
nucleotide polymorphism loci selected in East Asia populations

| Parameters | 677 SNPs | 258 SNPs |
|---|---|---|
| Accuracy | 0.9439 | 0.9459 |
| Kappa | 0.9297 | 0.9324 |

The above-mentioned embodiments only describe the preferred mode of the application, and do not limit the scope of the application. Under the premise of not departing from the design spirit of the application, various modifications and improvements made by ordinary technicians in the field to the technical scheme of the application shall fall within the protection scope determined by the claims of the application.

What is claimed is:

1. An application of a detection reagent of a group of whole-genome SNP loci used for identifying biogeographic origins of East Asian populations in preparing a kit for identifying the biogeographic origins of the East Asian populations, wherein the biogeographic origins of the East Asian populations is selected from Beijing Han population, Southern Han population, Dai population, Japanese and Kinh Population from Vietnam; the SNP loci comprise loci shown in a following table:

| chromosome | rs number | position | allele 1 | allele 2 |
|---|---|---|---|---|
| 1 | rs6594028 | 564598 | G | A |
| 1 | rs1801133 | 11856378 | A | G |
| 1 | rs12038287 | 11895396 | C | T |
| 1 | rs561510556 | 12387655 | A | G |
| 1 | rs144246431 | 19674993 | G | T |
| 1 | rs202129706 | 22315762 | A | C |
| 1 | rs140295961 | 33068395 | A | G |
| 1 | rs12731453 | 36676712 | T | G |
| 1 | rs117115434 | 56279497 | A | G |
| 1 | rs576196822 | 62612083 | T | C |
| 1 | rs532154984 | 65314266 | T | C |
| 1 | rs56270653 | 83804841 | C | G |
| 1 | rs552858520 | 84679675 | A | T |
| 1 | rs77172129 | 98602316 | G | A |
| 1 | rs147226864 | 121471638 | T | C |
| 1 | rs6692177 | 143543213 | A | G |
| 1 | rs200220063 | 152882512 | G | A |
| 1 | rs183624843 | 156665281 | T | C |
| 1 | rs16840204 | 158435927 | A | C |
| 1 | rs75985579 | 158988992 | A | G |
| 1 | rs75735370 | 187472432 | G | A |
| 1 | rs7530988 | 205558200 | G | A |
| 1 | rs151191827 | 229641396 | A | G |
| 1 | rs12726054 | 233623860 | A | G |
| 2 | rs77944863 | 3225405 | A | G |
| 2 | rs551794229 | 5162546 | A | G |
| 2 | rs187901830 | 32048491 | G | T |
| 2 | rs530416094 | 39536678 | A | G |
| 2 | rs75837024 | 48763333 | G | A |
| 2 | rs80297078 | 68051286 | C | T |
| 2 | rs557609484 | 92310281 | T | C |
| 2 | rs56339353 | 92320508 | C | A |
| 2 | rs114979404 | 97613974 | G | C |
| 2 | rs189257511 | 97718250 | T | A |
| 2 | rs143319605 | 103166662 | C | T |
| 2 | rs55935451 | 147238877 | A | T |
| 2 | rs55868911 | 177272945 | A | G |
| 2 | rs117736789 | 177439091 | C | G |
| 2 | rs537631083 | 210638066 | A | G |
| 2 | rs146508123 | 226363646 | T | C |
| 3 | rs59692692 | 13571964 | A | T |
| 3 | rs142773888 | 14414901 | T | C |
| 3 | rs144955067 | 31628063 | T | G |
| 3 | rs80350736 | 61914553 | T | C |
| 3 | rs79961039 | 68328083 | C | T |
| 3 | rs73107449 | 69415703 | C | T |
| 3 | rs77486591 | 69513520 | T | A |
| 3 | rs570435573 | 86028382 | T | G |
| 3 | rs544325853 | 97279356 | G | T |
| 3 | rs6778948 | 150134304 | G | A |
| 3 | rs11706245 | 150193109 | G | A |
| 3 | rs9844691 | 150250537 | C | A |
| 3 | rs116783706 | 152553769 | T | C |
| 3 | rs112658986 | 175079928 | C | A |
| 3 | rs575001940 | 183674928 | A | G |
| 3 | rs79806084 | 187520132 | C | T |
| 4 | rs142462241 | 9123223 | C | T |
| 4 | rs370496197 | 9240814 | T | C |
| 4 | rs546642722 | 17813761 | A | G |
| 4 | rs76753571 | 38787305 | G | A |
| 4 | rs5743592 | 38803063 | G | A |
| 4 | rs55750794 | 38851296 | T | C |
| 4 | rs55718051 | 38906717 | G | A |
| 4 | rs7680508 | 100445282 | G | A |
| 4 | rs9884555 | 120869851 | G | T |
| 4 | rs1425419 | 124565964 | T | C |
| 4 | rs280603 | 129915063 | C | A |
| 4 | rs17682978 | 137834738 | C | G |
| 5 | rs201981916 | 1025907 | T | C |
| 5 | rs12658612 | 31238976 | T | G |
| 5 | rs370349765 | 37295709 | T | C |
| 5 | rs78369336 | 41181491 | T | G |
| 5 | rs145999897 | 49432282 | A | G |
| 5 | rs28834498 | 49436826 | G | A |
| 5 | rs75712375 | 65307199 | A | T |
| 5 | rs3850651 | 88181109 | G | T |
| 5 | rs10066711 | 88190604 | T | A |
| 5 | rs117108524 | 88780333 | T | G |
| 5 | rs62381226 | 138366518 | T | C |
| 5 | rs4912927 | 142951094 | A | G |
| 5 | rs74562701 | 172998005 | A | G |
| 6 | rs75585369 | 5138833 | G | A |
| 6 | rs74567382 | 6183479 | A | G |
| 6 | rs56091651 | 14009167 | A | G |
| 6 | rs184103375 | 38488488 | T | C |
| 6 | rs62412779 | 58774684 | G | A |
| 6 | rs7766881 | 82802644 | C | A |
| 6 | rs2815293 | 96769927 | T | C |
| 6 | rs9480779 | 107836678 | C | T |
| 6 | rs565359437 | 108108169 | A | G |
| 6 | rs9402549 | 134239300 | C | T |
| 6 | rs4464817 | 138340676 | A | G |
| 6 | rs535319466 | 152588967 | G | C |
| 6 | rs9457053 | 165622609 | A | G |
| 6 | rs112864719 | 169342074 | A | C |
| 6 | rs75191948 | 170619277 | A | G |
| 7 | rs535914822 | 42834578 | G | C |
| 7 | rs141756608 | 50275516 | T | C |
| 7 | rs200588960 | 61794552 | T | A |
| 7 | rs374938140 | 61794862 | C | T |
| 7 | rs6958030 | 66457975 | C | T |
| 7 | rs76950224 | 130932529 | G | C |
| 7 | rs60560877 | 134697870 | A | G |
| 7 | rs10269898 | 141790229 | G | A |
| 7 | rs3778922 | 151802332 | T | G |
| 8 | rs144799228 | 4172014 | C | T |
| 8 | rs187561464 | 9673968 | A | G |
| 8 | rs117900444 | 32351714 | G | A |
| 8 | rs199569147 | 43825355 | G | T |
| 8 | rs62497902 | 46846688 | A | G |
| 8 | rs372912309 | 46846701 | A | C |
| 8 | rs77994895 | 80546112 | A | T |
| 8 | rs78475651 | 106445484 | G | C |
| 8 | rs80311821 | 119297519 | C | T |
| 8 | rs117673129 | 121843399 | A | G |
| 8 | rs4523256 | 123206335 | C | T |
| 8 | rs77058162 | 123624226 | C | T |
| 8 | rs117059004 | 123765817 | A | G |
| 8 | rs4736545 | 133114957 | A | C |
| 8 | rs2976388 | 143760256 | A | G |
| 9 | rs10816006 | 8937989 | G | T |
| 9 | rs1359095 | 10276100 | C | T |
| 9 | rs7039736 | 29819149 | A | G |
| 9 | rs117745218 | 34851653 | T | C |
| 9 | rs118138111 | 35388117 | C | T |
| 9 | rs117359308 | 44239346 | A | G |
| 9 | rs62547870 | 68396587 | C | T |
| 9 | rs117532342 | 123007609 | C | A |
| 9 | rs10760415 | 128892050 | A | G |
| 9 | rs3780712 | 132943082 | A | G |
| 10 | rs116843849 | 14693330 | T | C |
| 10 | rs58098705 | 25499954 | A | G |
| 10 | rs74213410 | 42399151 | A | T |
| 10 | rs192073133 | 43427620 | T | C |
| 10 | rs2339711 | 53048696 | G | A |
| 10 | rs1649994 | 80070687 | C | G |
| 10 | rs576091513 | 101292805 | G | T |
| 10 | rs75509020 | 134369277 | C | G |
| 11 | rs2071118 | 2972439 | T | C |
| 11 | rs4757893 | 20133413 | G | A |
| 11 | rs145321302 | 34240293 | C | G |
| 11 | rs12785447 | 38438330 | C | G |
| 11 | rs149709595 | 44840723 | C | T |
| 11 | rs1484393 | 45024657 | G | A |
| 11 | rs117641284 | 47248190 | G | A |
| 11 | rs11039176 | 47339169 | G | A |
| 11 | rs10838794 | 48054573 | T | C |
| 11 | rs11039516 | 48124157 | A | T |
| 11 | rs7941996 | 50496359 | T | C |
| 11 | rs147042619 | 60956757 | A | G |
| 11 | rs117682486 | 61015168 | C | T |
| 11 | rs11230736 | 61304473 | C | T |
| 11 | rs143362806 | 61375236 | G | T |
| 11 | rs520987 | 61521446 | C | A |
| 11 | rs7394579 | 61581450 | A | G |
| 11 | rs7394739 | 69692121 | T | C |
| 11 | rs74355568 | 114324060 | T | A |
| 11 | rs10891749 | 114647037 | C | T |
| 11 | rs80253223 | 118722457 | A | C |
| 11 | rs117608910 | 118741152 | C | T |
| 11 | rs189120206 | 119197644 | A | G |
| 11 | rs79626515 | 119980685 | A | G |
| 11 | rs11223547 | 133528942 | A | T |
| 12 | rs3217805 | 4388084 | G | C |
| 12 | rs429561 | 52835321 | C | G |
| 12 | rs77994613 | 54618848 | C | T |
| 12 | rs11170914 | 54861704 | C | T |
| 12 | rs10506426 | 61775492 | C | A |
| 12 | rs536701895 | 75343015 | A | G |
| 12 | rs79705698 | 88508258 | C | T |
| 12 | rs78062178 | 89304157 | G | A |
| 12 | rs11105124 | 89375909 | A | T |
| 12 | rs10860945 | 103539215 | C | T |
| 12 | rs11066427 | 113263909 | G | C |
| 12 | rs11608584 | 128051560 | T | C |
| 13 | rs7328200 | 28615133 | A | G |
| 13 | rs74984577 | 102518262 | T | A |
| 13 | rs540356754 | 113541917 | G | C |
| 14 | rs182863287 | 22445293 | C | T |
| 14 | rs2042518 | 76166481 | T | C |
| 14 | rs78964863 | 89771738 | G | C |
| 14 | rs144885709 | 95893762 | A | T |
| 14 | rs538254210 | 96938945 | T | A |
| 14 | rs77313258 | 101788844 | T | C |
| 14 | rs189231680 | 105862413 | A | T |
| 14 | rs77597431 | 106029023 | T | A |
| 14 | rs8003259 | 106063104 | T | G |
| 14 | rs4983473 | 106081193 | T | C |
| 14 | rs61985604 | 106085447 | C | T |
| 14 | rs75889359 | 106117651 | G | T |
| 14 | rs28720689 | 106127912 | G | A |
| 14 | rs10150934 | 106129418 | T | C |
| 14 | rs2516751 | 106143806 | G | A |
| 14 | rs7494172 | 106175202 | T | C |
| 14 | rs372579409 | 106185689 | C | G |
| 14 | rs186911060 | 106187159 | G | C |
| 14 | rs17841089 | 106207725 | C | T |
| 14 | rs12880412 | 106207805 | C | G |
| 14 | rs61983938 | 106210814 | T | C |
| 14 | rs140451109 | 106225946 | G | C |
| 14 | rs61985395 | 106231158 | G | A |
| 14 | rs2879250 | 106235419 | C | T |
| 14 | rs15979 | 106235489 | T | C |
| 14 | rs1051112 | 106235611 | A | T |
| 14 | rs149653267 | 106235742 | C | G |
| 14 | rs12101008 | 106340358 | T | A |
| 15 | rs12050504 | 25118733 | C | T |
| 15 | rs8038186 | 56095508 | A | G |
| 15 | rs117054397 | 60472480 | A | G |
| 15 | rs370188878 | 60756638 | G | A |
| 15 | rs2439424 | 66979943 | A | G |
| 15 | rs536189723 | 74326699 | C | T |
| 15 | rs558029138 | 101098151 | C | A |
| 16 | rs570636147 | 16452036 | C | T |
| 16 | rs4275872 | 46410819 | G | A |
| 16 | rs543086096 | 46417894 | A | G |
| 16 | rs9285998 | 46426086 | G | A |
| 16 | rs17822931 | 48258198 | C | T |
| 16 | rs7185374 | 48450368 | C | A |
| 16 | rs148106276 | 87864696 | T | C |
| 16 | rs55799444 | 90107716 | T | C |
| 17 | rs76007934 | 2371207 | C | G |
| 17 | rs142708997 | 21965750 | T | C |
| 17 | rs141797564 | 22253602 | T | G |
| 17 | rs202121576 | 22261435 | C | T |
| 17 | rs79399637 | 22261755 | G | T |
| 17 | rs139316749 | 22262103 | T | A |
| 17 | rs78261308 | 36778892 | C | A |
| 17 | rs75060014 | 41038677 | A | G |
| 17 | rs147994591 | 45627005 | A | G |
| 17 | rs140713446 | 46124685 | C | G |
| 17 | rs140900296 | 47089580 | G | T |
| 17 | rs6501525 | 70218627 | A | G |
| 17 | rs77039319 | 70278839 | A | G |
| 17 | rs189618173 | 73722924 | T | C |
| 18 | rs545537217 | 18518431 | T | G |
| 18 | rs6567282 | 60094992 | C | T |
| 19 | rs8100854 | 10720886 | A | T |
| 19 | rs10408721 | 10758319 | T | C |
| 19 | rs138357154 | 17601811 | T | C |
| 19 | rs12986064 | 54755133 | C | T |
| 19 | rs624315 | 54755636 | T | C |
| 19 | rs377681 | 54766423 | A | G |
| 19 | rs1808548 | 54781509 | T | C |
| 19 | rs798899 | 54800767 | T | C |
| 20 | rs6117562 | 753310 | G | A |
| 20 | rs6140211 | 773680 | G | A |
| 20 | rs565751489 | 5547557 | T | A |
| 20 | rs118072189 | 26292074 | T | G |
| 21 | rs59142554 | 35544523 | A | G |
| 21 | rs549950103 | 38533018 | A | T |
| 21 | rs114285135 | 41457206 | C | A |
| 22 | rs540495340 | 20663250 | A | C |
| 22 | rs148969952 | 30958591 | G | C |
| 22 | rs57437434 | 37373430 | A | C |
| 22 | rs138225077 | 42121201 | T | C |
| 22 | rs117410509 | 48654537 | T | C |
| 22 | rs551265777 | 49277658 | G | C |

2. A method for analyzing biogeographic origins of East Asian populations, comprising steps of screening the group of whole-genome SNP loci for identifying the biogeographic origins of the East Asian populations according to claim 1.

3. The method according to claim 2, wherein following steps are specifically comprised:
(1) based on whole-genome data of the East Asian populations in international 1,000 genomes, using a PLINK software system to preliminarily screen relatively highly differentiated SNP loci in the East Asian populations; and
(2) using an XGBoot machine learning algorithm, re-screening the SNP loci preliminarily screened in the step (1) based on an optimal subset method, and finally determining the SNP loci used to analyze the biogeographic origins of the East Asian populations.

4. The method according to claim 3, wherein in the step (1), principles of preliminarily screening relatively highly differentiated SNP loci in the East Asian populations comprise:
(1) fixed coefficients of the Japanese and a non-Japanese population greater than 0.2;
(2) fixed coefficients of the Beijing Han population and the Southern Han population greater than 0.06;
(3) fixed coefficients of the Dai population and the Kinh Population from Vietnam greater than 0.06;
(4) fixed coefficients of a Han population, the Dai population, and the Kinh Population greater than 0.06;
(5) a minimum allele frequency of selected SNP loci in each population greater than 0.01;
(6) the selected SNP loci consistent with HWE in each population, and a P value greater than 0.0001; and
(7) paired r² of the selected SNP loci less than 0.6.

5. The method according to claim 3, wherein between the step (1) and the step (2) further comprising: using a principal component analysis method to evaluate an analytic efficiency of the SNP loci preliminarily screened in the step (1) on the East Asian populations.

6. The method according to claim 3, wherein further comprising: using the re-screened SNP loci obtained in the step (2) to construct a prediction model, and evaluating an identification efficiency on the biogeographic origins of the East Asian populations.

7. An application of the group of whole-genome SNP loci for identifying the biogeographic origins of the East Asian populations according to claim 1 in population genetics research.
